(19) United States
(12) Patent Application Publication
(10) Pub. No.: US 2025/0318710 A1
(43) Pub. Date: Oct. 16, 2025

(54) VARIABLE OFFSET BUTTON FOR ENDOSCOPE VALVES

(71) Applicant: Loma Linda University, Lomda Linda, CA (US)

(72) Inventor: Gautam Nagendra, Loma Linda, CA (US)

(21) Appl. No.: 19/177,317

(22) Filed: Apr. 11, 2025

Related U.S. Application Data
(60) Provisional application No. 63/633,030, filed on Apr. 11, 2024.

(51) Int. Cl. A61B 1/00; A61B 1/015

(52) U.S. Cl. CPC A61B 1/00068; A61B 1/00103; A61B 1/015

(57) ABSTRACT

Disclosed herein is an endoscope system. The endoscope system includes an insertion tube, a body comprising a plurality of valve cylinders, and a plurality of valves for insertion into one of the plurality of valve cylinders. Each of the plurality of valves comprises a button for depression along a depression axis, a spring extending along a vertical axis that is substantially parallel to the depression axis, an extension member extending along the vertical axis, and a neck to offset the button from the vertical axis. The neck is coupled to the button and the extension member such that depression of the button moves the neck and compresses the spring to move the extension member. The offset of the button of the plurality of valves facilitates improving one or more ergonomic conditions of a hand grip of an operator of the endoscope system.

### RELATED APPLICATIONS

The present disclosure claims priority to U.S. Provisional Application No. 63/633,030 entitled VARIABLE OFFSET BUTTON FOR ENDOSCOPE VALVES and filed on Apr. 11, 2024; the entire contents of which are hereby incorporated by reference.

### FIELD

The present disclosure relates to endoscopes for use in medical procedures. In particular, the present disclosure relates to an endoscope device with valve buttons positioned for improved ergonomic operation.

### BACKGROUND

Endoscopes are devices that contain a forward viewing camera at the end of a flexible tube and are used in multiple fields for minimally invasive procedures. Many endoscopy procedures utilize a reusable endoscope device, often including a single use, disposable valve set for each procedure. A valve set may include an air/water valve and a suction valve, each of which may include a button. During operation, the button of the air/water valve and/or the suction valve may be depressed to activate the valve. For example, depression of the air/water valve button may allow for water flow at the distal tip of the flexible tube depending on the degree of button depression. Additionally, for example, depression of the suction valve button may initiate suction of fluids and debris at the distal tip of the endoscope.

Many endoscopy procedures may require operators to exert high pinch forces, perform repetitive hand motions, endure contact stress, and/or maintain awkward postures while operating the endoscope. This may increase an operator's risk of sustaining work-related musculoskeletal injuries, including, but not limited to, pain in the thumb, neck, back, and/or shoulder(s). Furthermore, the risk of sustaining injury may be increased for an operator with a smaller hand size. Thus, there is a need for disposable valves with buttons that are positioned for improved ergonomic operation of an endoscope device.

### SUMMARY

Some aspects of the present disclosure provide an endoscope system and methods of use thereof. One aspect of the present disclosure includes an endoscope system including an insertion tube, a body including a plurality of valve cylinders, and a plurality of valves for insertion into one of the plurality of valve cylinders. Each of the plurality of valves includes a button for depression along a depression axis, a spring extending along a vertical axis that is substantially parallel to the depression axis, an extension member extending along the vertical axis, and a neck to offset the button from the vertical axis. The neck is coupled to the button and the extension member such that depression of the button moves the neck and compresses the spring to move the extension member. The offset of the button of the plurality of valves facilitates improving one or more ergonomic conditions of a hand grip of an operator of the endoscope system.

Another aspect of the present disclosure includes a method of using an endoscope system. The endoscope system includes an insertion tube, a body comprising a plurality of valve cylinders, and a plurality of valves for insertion into one of the plurality of valve cylinders. The method includes inserting the plurality of valves into the plurality of valve cylinders, each of the plurality of valve cylinders including a button for depression along a depression axis, a spring extending along a vertical axis that is substantially parallel to the depression axis, an extension member extending along the vertical axis, and a neck to offset the button from the vertical axis. The neck is coupled to the button and the extension member such that depression of the button moves the neck and compresses the spring to move the extension member. The method also includes inserting a distal end of the insertion tube into a patient, advancing the distal end of the insertion tube to a desired location within the patient, and operating the endoscope system. The offset of the button of the plurality of valves facilitates improving one or more ergonomic conditions of a hand grip of an operator of the endoscope system. The method further includes removing the insertion tube from the patient.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A and 1B are perspective views of an endoscope device as known in the prior art.

FIGS. 2A and 2B are perspective and side views of a suction valve for use with an endoscope device as known in the prior art.

FIG. 2C is a schematic of depressing a button of the suction valve shown in FIG. 2A.

FIG. 2D is a bottom view of the suction valve shown in FIG. 2A.

FIG. 2E is a top view of a suction cylinder of an endoscope device into which the suction valve shown in FIG. 2A is inserted when in use.

FIGS. 3A and 3B are perspective and side views of an air/water valve for use with an endoscope device as known in the prior art.

FIG. 3C is a schematic of covering and depressing a button of the air/water valve shown in FIG. 3A.

FIGS. 4A and 4B are perspective views of an operator using an endoscope device as known in the prior art.

FIG. 4C is a side view of an example difference in hand size.

FIG. 4D is a perspective view of an operator with a smaller hand size using an endoscope device as known in the prior art.

FIG. 5A is a schematic of a side view of a suction valve for use with an endoscope device according to one or more embodiments, the suction valve including a button positioned for improved ergonomic operation.

FIG. 5B is a schematic of a top view of the suction valve shown in FIG. 5A according to one or more embodiments.

FIG. 5C is a schematic of a bottom view of the suction valve shown in FIG. 5A according to one or more embodiments.

FIG. 6A is a schematic of a side view of an air/water valve for use with an endoscope device according to one or more embodiments, the air/water valve including a button positioned for improved ergonomic operation.

FIG. 6B is a schematic of a top view of the air/water valve shown in FIG. 6A according to one or more embodiments.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

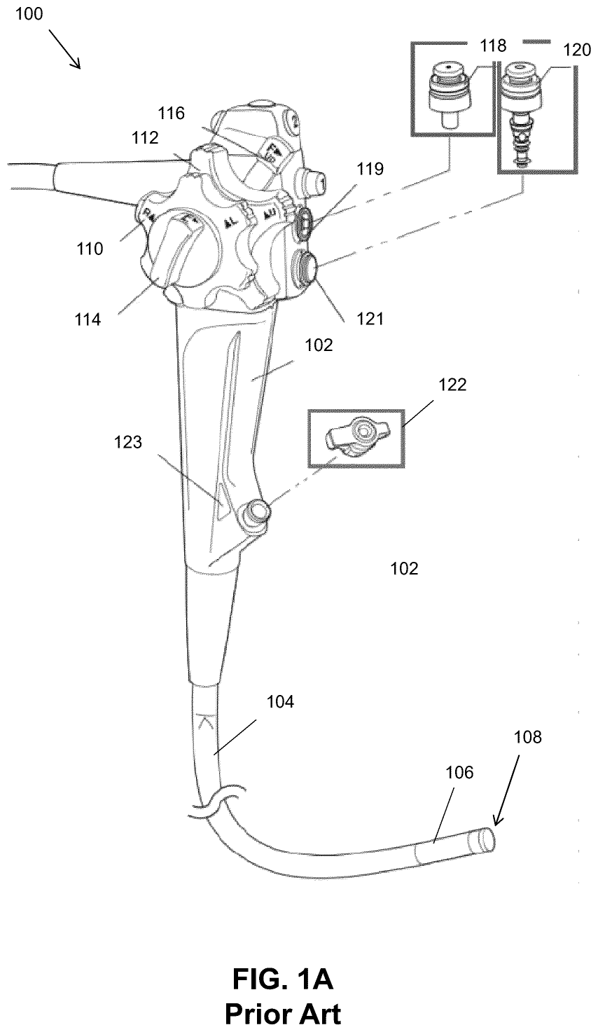

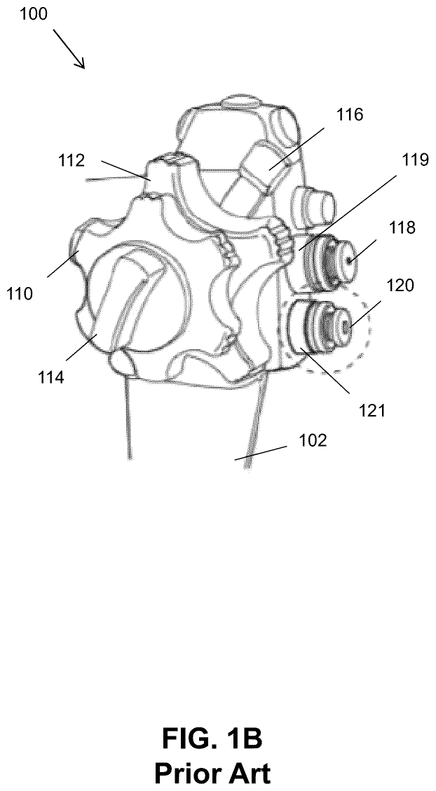

FIGS. 1A and 1B are perspective views of an endoscope device 100 as known in the prior art. The endoscope device includes a body 102 and an insertion tube 104. The body 102 includes one or more knobs to control movement of a bendable tube segment 106 at a distal end 108 of the insertion tube 104, including a first angulation control knob 112 to control up/down movement and a second angulation control knob 110 to control right/left movement. The body 102 also includes a first lock 114 and a second lock 116 to lock the first and second angulation control knobs 110, 112, respectively, in place, thereby locking the position of the bendable tube segment 106 of the insertion tube 104.

The body 102 also includes one or more valve cylinders for insertion of one or more valves, including a suction valve 118 for insertion in a suction cylinder 119, an air/water valve 120 for insertion in an air/water cylinder 121, and a biopsy valve 122 for insertion in a biopsy cylinder 123. The endoscope device 100 may be designed for reusability, with the suction valve 118, the air/water valve 120, and/or the biopsy valve 122 being disposable and thus intended for a single use via insertion and removal from the respective valve cylinder of the body 102.

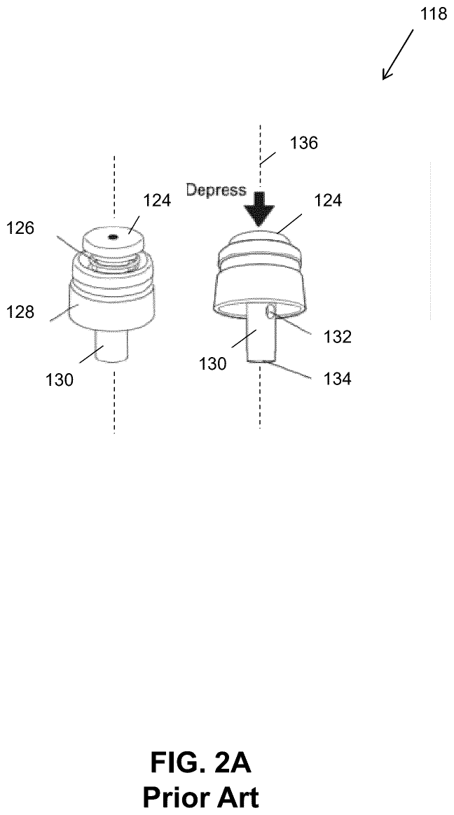

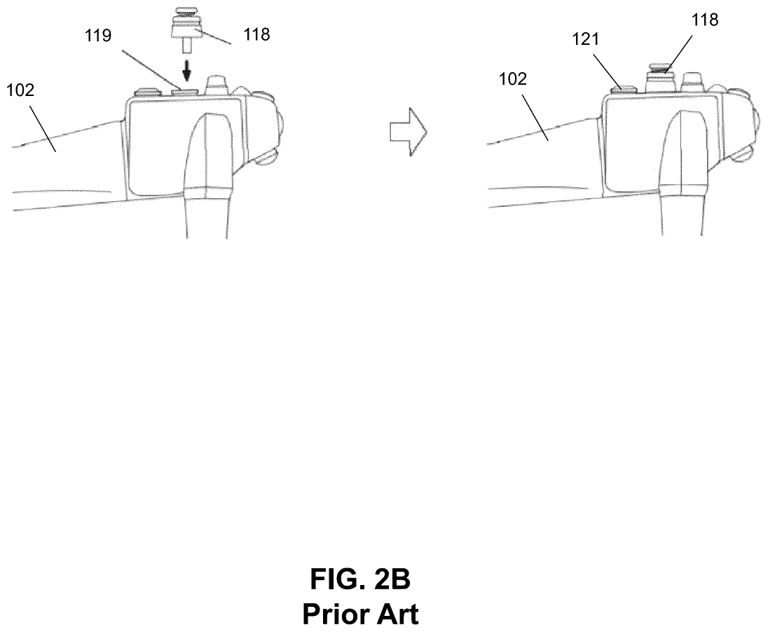

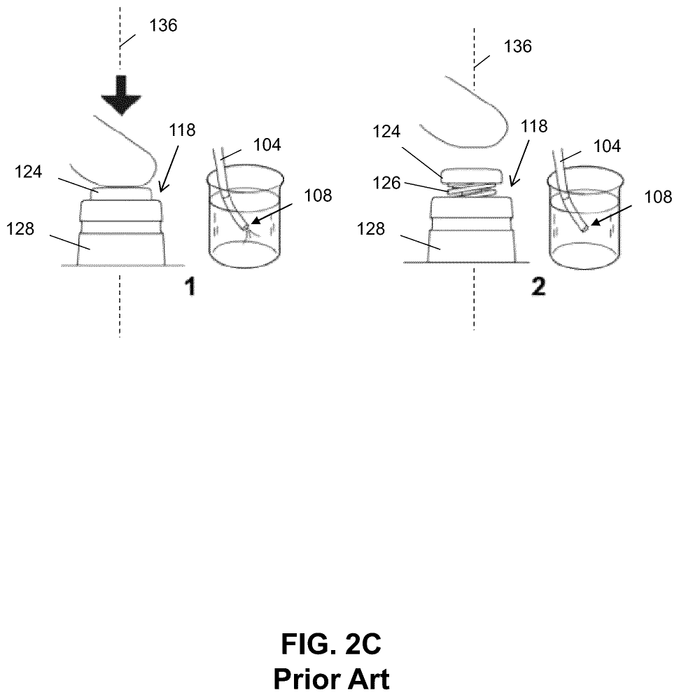

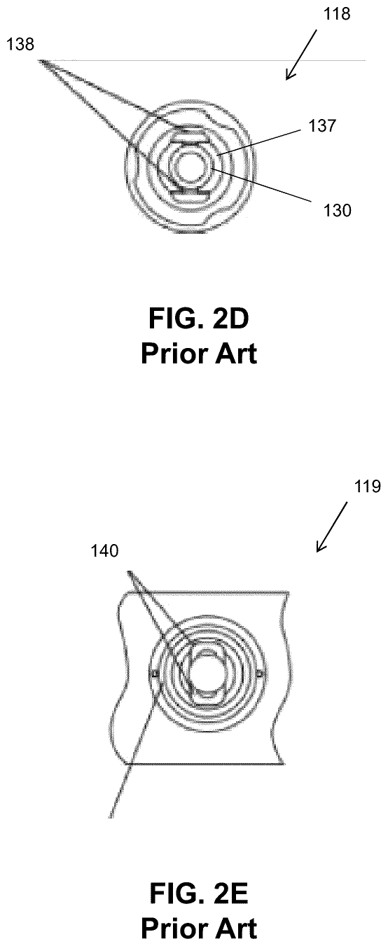

FIG. 2A is a perspective view, and FIG. 2B is a side view, of the suction valve 118 for use with the endoscope device 100. The suction valve 118 includes a button 124, a spring 126, a skirt 128, and an extension member 130 coupled to the button 124.

The extension member 130 includes a side hole 132 and a bottom hole 134. The button 124 is intended for engagement with an operator of the endoscope device 100, specifically a finger of the operator as described further herein.

FIG. 2C is a schematic of depressing the button 124 of the suction valve 118. Depression of the button 124, and thereby the spring 126, moves the extension member 130 within the skirt 128 along a depression axis 136 to enable the suction valve 118 to suction fluid from the distal end 108 of, and through, the insertion tube 104.

FIG. 2D is a bottom view of the suction valve 118 and FIG. 2E is a top view of the suction cylinder 119. The suction valve 118 includes a retaining ring 137 positioned proximate the spring 126. When the button 124 is depressed to compress the spring 126 and move the extension member 130, the retaining ring 137 remains stationary. The retaining ring 137 includes a pair of ridges 138 protruding outwards (e.g., away from the extension member 130). When the suction valve 118 is inserted into the suction cylinder 119, the ridges 138 of the retaining ring 137 are aligned with a pair of secondary ridges 140 of the suction cylinder 119 to facilitate proper insertion of the suction valve 118.

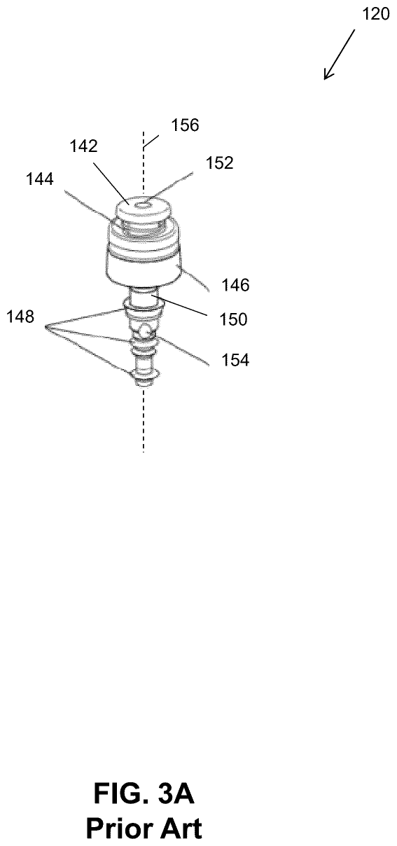

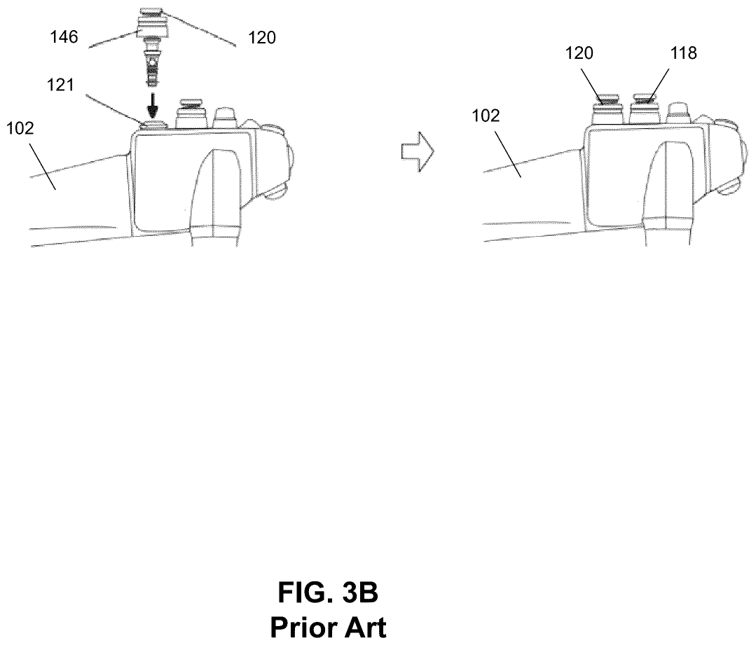

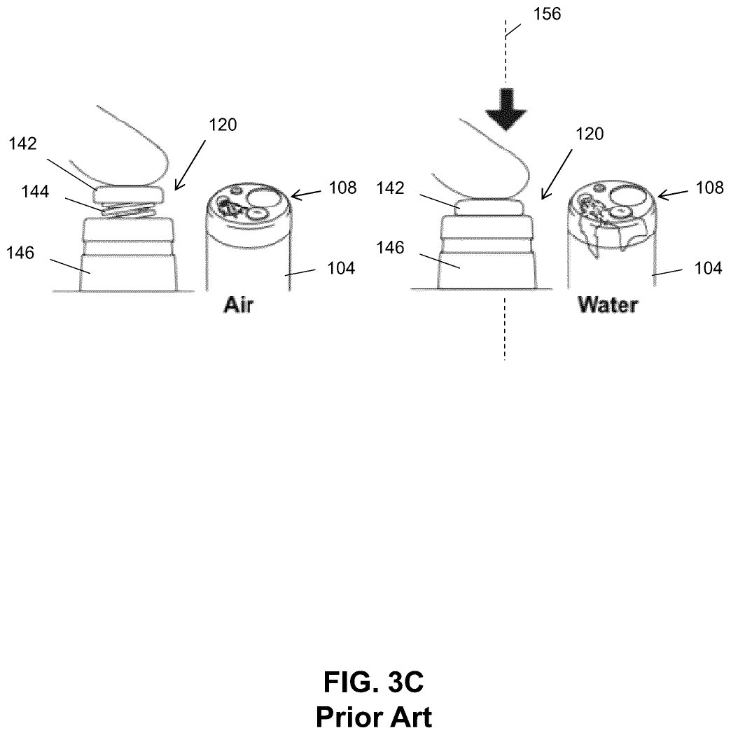

FIG. 3A is a perspective view, and FIG. 3B is a side view, of the air/water valve 120 for use with the endoscope device 100. The air/water valve 120 includes a button 142, a spring 144, a skirt 146, a plurality of seals 148, and an extension member 150 coupled to the button 142. The button 142 includes a top hole 152 and the extension member 150 includes a side hole 154, the top hole 152 and the side hole 154 being in continuity. The button 142 is intended for engagement with an operator of the endoscope device 100, specifically a finger of the operator as described further herein.

FIG. 3C is a schematic of covering and depressing the button 142 of the air/water valve 120. Depression of the button 142, and thereby the spring 144, moves the extension member 150 within the skirt 146 along a depression axis 156 to enable the air/water valve 120 to activate water flow at the distal end 108 of the insertion tube 104. Additionally, covering the top hole 152 of the button 142 allows a flow of air to be redirected to, and out of, the distal end 108 of the insertion tube 104. The plurality of seals 148 are positioned such that an air channel remains open throughout operation of the air/water valve 120, with a path of least resistance for air flow changing depending on whether the top hole 152 of the button 142 is covered. The air/water valve 120 includes a retaining ring (not shown) positioned proximate the spring 144. When the button 142 is depressed to compress the spring 144 and move the extension member 150, the retaining ring (not shown) remains stationary.

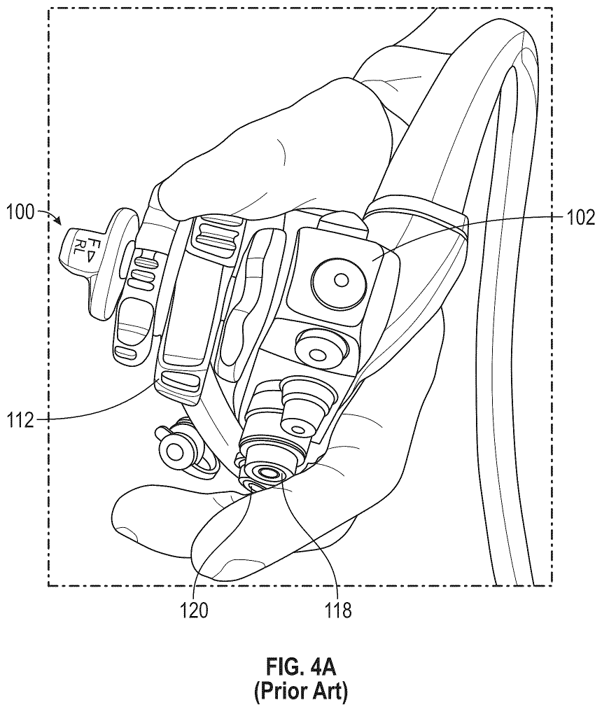

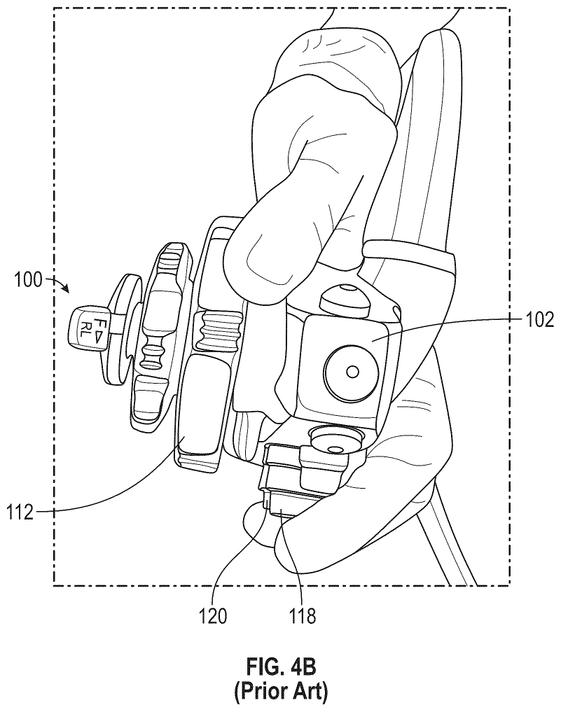

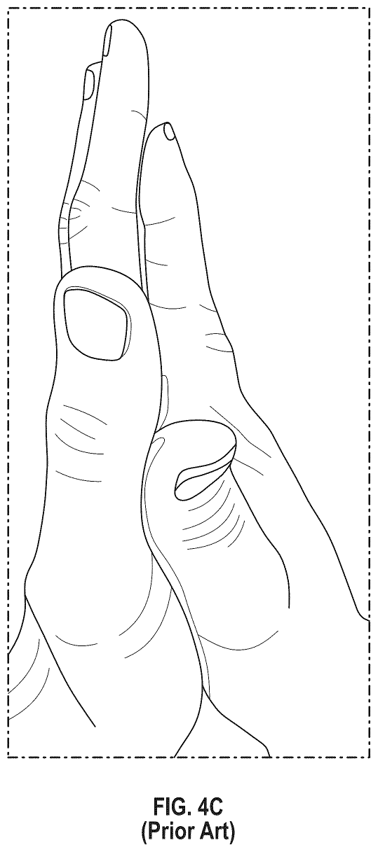

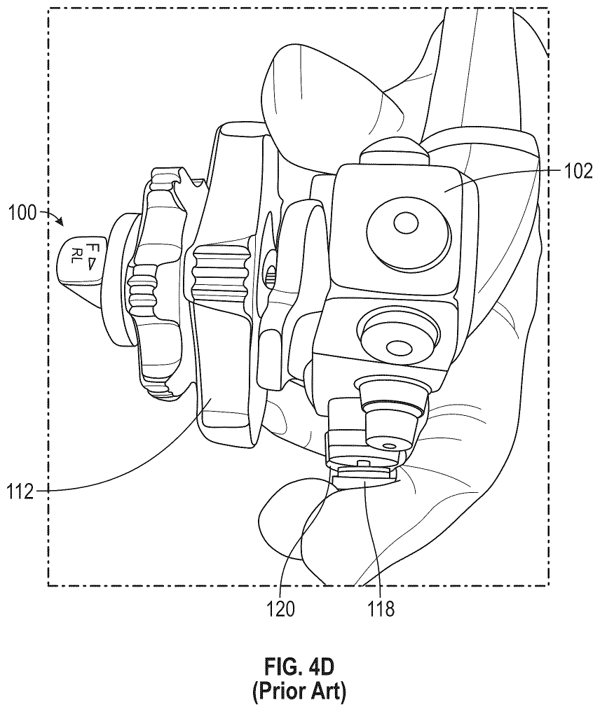

FIGS. 4A and 4B are perspective views of an operator using the endoscope device 100. As shown in FIG. 4A, a resting grip of a hand of an operator may include a thumb on the first angulation control knob 112, an index finger extending past the suction valve 118, and a middle finger extending past the air/water valve 120. As shown in FIG. 4B, an active grip of a hand of an operator to use the endoscope device 100 may include the thumb on the first angulation control knob 112, the index finger in a semi-clawed position on the suction valve 118, and the middle finder in a semi-clawed position on the air/water valve 120. The semi-clawed position of the fingers in the active grip may require the operator to exert high pinch forces, endure contact stress, and/or maintain awkward postures while operating the endoscope device 100. This may increase the operator's risk of sustaining work-related musculoskeletal injuries.

FIG. 4C is a side view illustrating an example difference in hand size. FIG. 4D is a perspective view of an operator with a smaller hand size using the endoscope device 100. As shown in FIG. 4D, an active grip of a hand of an operator with a smaller hand size to use the endoscope device 100 may include the thumb on the body 102, the index finger in a minimal semi-clawed position on the suction valve 118, and the middle finder in a minimal semi-clawed position on the air/water valve 120. The required reach of the thumb to move to the first angulation control knob 112 while keeping the minimal semi-clawed position of the index and middle fingers may increase the operator's risk of sustaining work-related musculoskeletal injuries.

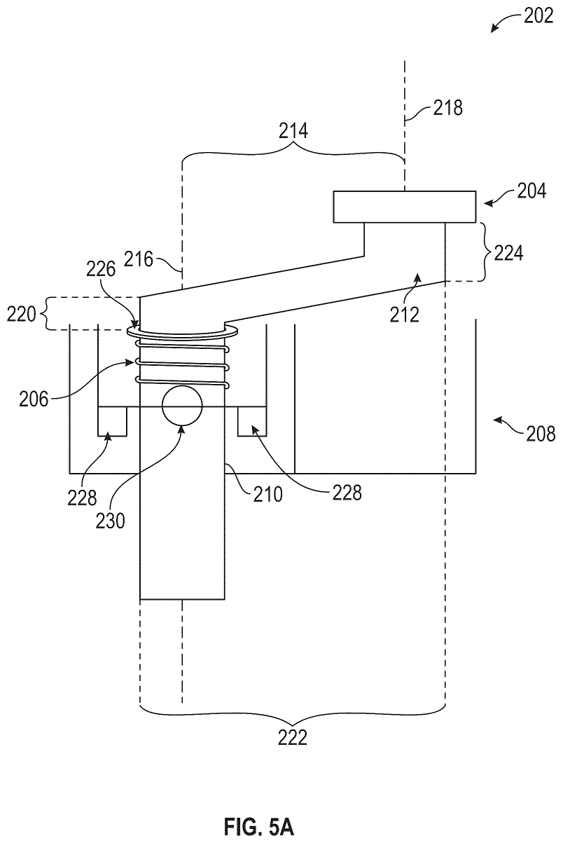

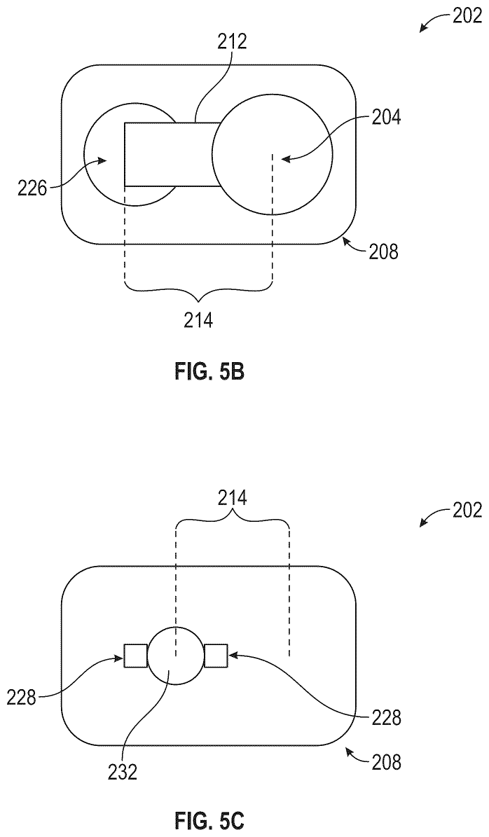

FIG. 5A is a schematic of a side view of a suction valve 202 for use with an endoscope device, such as, but not limited to, the endoscope device 100. The suction valve 202 includes a button 204, a spring 206, a skirt 208, an extension member 210, and a neck 212. The suction valve 200 differs from the suction valve 118 in that the suction valve 200 includes the neck 212 that is coupled to the button 204 and the extension member 210, the neck 212 positioning the button 204 for improved ergonomic operation of the endoscope device. Specifically, the button 204 is positioned at an offset 214 from a vertical axis 216 extending through the spring 206. The vertical axis 216 is substantially parallel to a depression axis 218 of the button 204.

The neck 212 may be sized and/or shaped for the offset 214 of the button 204 from the vertical axis 216 to be up to one inch. As shown in FIG. 5A, the neck 212 may include a first vertical segment 220, a horizontal segment 222, and a second vertical segment 224. In some embodiments, the segments of the neck 212 may all be of a single unitary structure. In other embodiments, the segments of the neck 212 may be individual components that are assembled or connected to form the complete structure. Additionally, or alternatively, to the offset 214 positioning of the button 204, the button 204 may be positioned such that the depression axis 218 is at an angle relative to the vertical axis 216 for improved ergonomic operation of the endoscope device. In some embodiments, the neck 212 may be curved, such that the junctions between the first vertical segment 220, the horizontal segment 222, and the second vertical segment 224 are rounded.

The neck 212 is coupled to the button 204 and the extension member 210, such that depression of the button 204 moves the neck 212 and thereby compresses the spring 206 and moves the extension member 210 within the skirt 208. The movement of the extension member 210 along the vertical axis 216 enables the suction valve 202 to initiate suction at a distal end of an insertion tube of an endoscope device, such as the distal end 108 of the insertion tube 104. The neck 212 may be coupled to a top ring 226 with a radius slightly larger than a radius of the spring 206 to translate the depression movement of the button 204 to compression of the spring 206. In some embodiments, a radius of the neck 212, specifically the first vertical segment 220 of the neck 212, may be slightly larger than the radius of the spring 206 to perform the movement translation function of a top ring at the coupling of the neck 212 to the spring 206. The size and/or shape of the neck 212 may require a size and/or spring constant of the spring 206 to be adjusted to maintain the degree of depression.

FIG. 5B is a top view, and FIG. 5C is a bottom view, of the suction valve 202 for use with an endoscope device, such as, but not limited to, the endoscope device 100. The suction valve 202 includes a retaining ring (not shown) positioned proximate the spring 206. When the button 204 is depressed to compress the spring 206 and move the extension member 210, the retaining ring (not shown) remains stationary. The retaining ring (not shown) includes a pair of ridges 228 protruding outwards (e.g., away from the extension member 210). When the suction valve 202 is inserted into a suction cylinder, such as the suction cylinder 119, the ridges 228 of the retaining ring (not shown) are aligned with a pair of secondary ridges of the suction cylinder, such as the pair of secondary ridges 140 of the suction cylinder 119, to facilitate proper insertion of the suction valve 202. The extension member includes a side hole 230 and a bottom hole 232 that are in continuity.

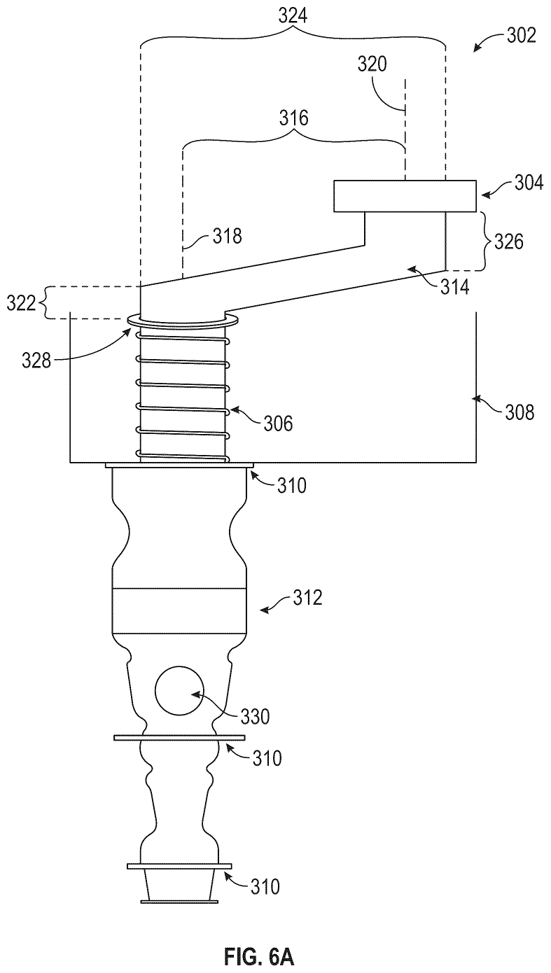

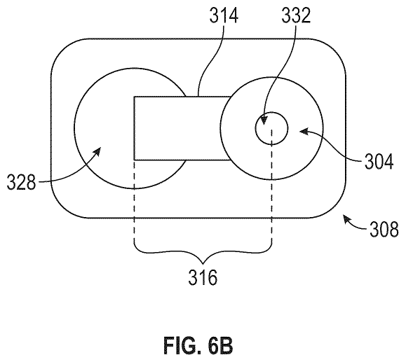

FIG. 6A is a schematic of a side view, and FIG. 6B is a top view, of an air/water valve 302 for use with an endoscope device, such as, but not limited to, the endoscope device 100. The air/water valve 302 includes a button 304, a spring 306, a retaining ring (not shown), a skirt 308, a plurality of seals 310, an extension member 312, and a neck 314. The air/water valve 302 differs from the air/water valve 120 in that the air/water valve 302 includes the neck 314 that is coupled to the button 304 and the extension member 312, the neck 314 positioning the button 304 for improved ergonomic operation of the endoscope device. Specifically, the button 304 is positioned at an offset 316 from a vertical axis 318 extending through the spring 306. The vertical axis 318 is substantially parallel to a depression axis 320 of the button 304.

The neck 314 may be sized and/or shaped for the offset 316 of the button 304 from the vertical axis 318 to be up to one inch. As shown in FIG. 6A, the neck 314 may include a first vertical segment 322, a horizontal segment 324, and a second vertical segment 326. In some embodiments, the segments of the neck 314 may all be of a single unitary structure. In other embodiments, the segments of the neck 314 may be individual components that are assembled or connected to form the complete structure. Additionally, or alternatively, to the offset 316 positioning of the button 304, the button 304 may be positioned such that the depression axis 320 is at an angle relative to the vertical axis 318 for improved ergonomic operation of the endoscope device.

The neck 314 is coupled to the button 304 and the extension member 312, such that depression of the button 304 moves the neck 314 and thereby compresses the spring 306 and moves the extension member 312 within the skirt 308. The movement of the extension member 312 along the vertical axis 318 allows the air/water seal 302 to activate water flow at a distal end of an insertion tube of an endoscope device, such as the distal end 108 of the insertion tube 104. The neck 314 may be coupled to a retaining ring 328 with a radius slightly larger than a radius of the spring 306 to translate the depression movement of the button 304 to compression of the spring 306. In some embodiments, a radius of the neck 314, specifically the first vertical segment 322 of the neck 314, may be slightly larger than the radius of the spring 306 to act as a top ring at the coupling of the neck 314 to compress the spring 306. The size and/or shape of the neck 314 may require a size and/or spring constant of the spring 306 to be adjusted to maintain the degree of depression. The extension member includes a side hole 330. The button 304 includes a top hole 332. Covering the top hole 332 of the button 304 enables a flow of air to be redirected to, and out of, a distal end of an insertion tube of an endoscope device, such as the distal end 108 of the insertion tube 104. When the button 304 is depressed to compress the spring 306 and move the extension member 312, the retaining ring 328 remains stationary.

The degree of offset may be the same or different between the suction valve 202 and the air/water valve 302. In some embodiments, the offset 214 of the suction valve 202 may be substantially similar to the offset 316 of the air/water valve 302. In other embodiments, the offset 214 of the suction valve 202 may be different than the offset 316 of the air/water valve 302. Additionally, in some embodiments, the offset 214 of the suction valve 202 and/or the offset 316 of the air/water valve 302 may include only a horizontal distance. In other embodiments, the offset 214 of the suction valve 202 and/or the offset 316 of the air/water valve 302 may include a horizontal distance and an angle relative to a horizontal axis. The degree of offset may also depend on a hand size of an operator of an endoscope device, such as, but not limited to, the endoscope device 100. That is, the offset 214 of the suction valve 202 and/or the offset 316 of the air/water valve 302 may vary depending on the hand size of the operator. In some embodiments, the neck 212/314 may be adjustable such that a height, a length, and/or one or more angles of the neck 212/314 may be varied. In other embodiments, multiple sizes of the neck 212/314 may be available for use with the device, such as one or more replaceable necks 212/314 of varying height, length, and/or angle. In still other embodiments, multiple sizes of one or more additional elements may be available for use with the device, such as one or more replaceable buttons 204/304 and/or springs 206/306 of varying height and/or length.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. An endoscope system comprising:
an insertion tube;
a body comprising a plurality of valve cylinders; and
a plurality of valves for insertion into one of the plurality of valve cylinders, each of the plurality of valves comprising:
a button for depression along a depression axis;
a spring extending along a vertical axis that is substantially parallel to the depression axis;
an extension member extending along the vertical axis; and
a neck to offset the button from the vertical axis,
wherein the neck is coupled to the button and the extension member such that depression of the button moves the neck and compresses the spring to move the extension member;
wherein the offset of the button of the plurality of valves facilitates improving one or more ergonomic conditions of a hand grip of an operator of the endoscope system.

2. The endoscope system of claim 1, wherein the plurality of valves comprises a suction valve, the depression of the button along the depression axis moving the extension member along the vertical axis to enable the suction valve to suction fluid into a distal end of the insertion tube.

3. The endoscope system of claim 1, wherein the plurality of valves comprises an air/water valve, the depression of the button along the depression axis moving the extension member along the vertical axis to enable the air/water valve to activate water flow at a distal end of the insertion tube.

4. The endoscope system of claim 3, wherein the button of the air/water valve comprises a hole and covering the hole enables a flow of air to be redirected to, and out of, the distal end of the insertion tube.

5. The endoscope system of claim 3, wherein the air/water valve comprises a plurality of seals protruding from the extension member, the movement of the extension member engaging one or more of the plurality of seals.

6. The endoscope system of claim 1, wherein the offset is up to one inch along a horizontal axis.

7. The endoscope system of claim 1, wherein the offset varies depending on a hand size of the operator of the endoscope system.

8. The endoscope system of claim 1, wherein the offset includes at least one of a horizontal distance and an angle relative to a horizontal axis.

9. The endoscope system of claim 1, wherein one or more of the plurality of valves are disposable and thus intended for a single use.

10. The endoscope system of claim 1, wherein the one or more ergonomic conditions comprise at least one of a force exertion, a contact stress, and a posture.

11. A method of using an endoscope system, the endoscope system comprising an insertion tube, a body comprising a plurality of valve cylinders, and a plurality of valves for insertion into one of the plurality of valve cylinders, the method comprising:
inserting the plurality of valves into the plurality of valve cylinders, each of the plurality of valves comprising:
a button for depression along a depression axis;
a spring extending along a vertical axis that is substantially parallel to the depression axis;
an extension member extending along the vertical axis; and
a neck to offset the button from the vertical axis, wherein the neck is coupled to the button and the extension member such that depression of the button moves the neck and compresses the spring to move the extension member;
inserting a distal end of the insertion tube into a patient;
advancing the distal end of the insertion tube to a desired location within the patient;
operating the endoscope system, wherein the offset of the button of the plurality of valves facilitates improving one or more ergonomic conditions of a hand grip of an operator of the endoscope system; and
removing the insertion tube from the patient.

12. The method of claim 11, wherein the plurality of valves comprises a suction valve, the depression of the button along the depression axis moving the extension member along the vertical axis to enable the suction valve to suction fluid into the distal end of the insertion tube.

13. The method of claim 11, wherein the plurality of valves comprises an air/water valve, the depression of the button along the depression axis moving the extension member along the vertical axis to enable the air/water valve to activate water flow at the distal end of the insertion tube.

14. The method of claim 13, wherein the button of the air/water valve comprises a hole and covering the hole enables a flow of air to be redirected to, and out of, the distal end of the insertion tube.

15. The method of claim 13, wherein the air/water valve comprises a plurality of seals protruding from the extension member, the movement of the extension member engaging one or more of the plurality of seals.

16. The method of claim 11, wherein the offset is up to one inch along a horizontal axis.

17. The method of claim 11, wherein the offset varies depending on a hand size of the operator of the endoscope system.

18. The method of claim 11, wherein the offset includes at least one of a horizontal distance and an angle relative to a horizontal axis.

19. The method of claim 11, wherein one or more of the plurality of valves are disposable and thus intended for a single use.

20. The method of claim 11, wherein the one or more ergonomic conditions comprise at least one of a force exertion, a contact stress, and a posture.

21. An endoscope system comprising at least one element or component described, in any order or combination.

22. A method of using an endoscope system, the method comprising at least one element or component described, in any order or combination.
